(19) United States
(12) Patent Application Publication
(10) Pub. No.: US 2015/0296760 A1
(43) Pub. Date: Oct. 22, 2015

(54) ROTATING FEEDER BIN FOR GROWING, FEDING AND HARVESTING INSECT LARVAE

(71) Applicant: Douglas A. Perednia, Portland, OR (US)

(72) Inventor: Douglas A. Perednia, Portland, OR (US)

(21) Appl. No.: 14/689,949

(22) Filed: Apr. 17, 2015

Related U.S. Application Data
(60) Provisional application No. 61/982,187, filed on Apr. 21, 2014.

(51) Int. Cl. A01K 67/033

(52) U.S. Cl. CPC A01K 67/033

(57) ABSTRACT

A system of feeding, culturing and harvesting insects, particularly black soldier flies comprises of a semi-enclosed barrel including a generally cylindrical sidewall having two ends and a central axis of rotation oriented roughly transversely to the direction of gravity, and a migration path attached to one or both ends of the barrel for the exit of mature larvae. A method of raising insect larvae and byproducts comprises of inserting larvae or eggs into a bin; comminuting and inserting food material into the interior of the bin; aerating the larval/food mass by rotating the bin about a central axis that is positioned roughly transverse to the direction of gravity; draining leachate from the bin into a collection system; and providing a migration path for the egress of mature larvae on at least one end of the bin into a larval collection system.

### CROSS-REFERENCES TO RELATED APPLICATIONS

This application claims benefit of U.S. Provisional Application No. 61/982,187, filed Apr. 21, 2014, titled ROTATING FEEDER BIN FOR GROWING, FEEDING AND HARVESTING INSECT LARVAE, which is incorporated herein by reference in its entirety.

### BACKGROUND

The present invention relates generally to systems for containing, feeding, growing and harvesting the larvae of a variety of potentially useful insect species such as the black soldier fly (BSF).

The black soldier fly (Hermetia illucens) is known to be a useful and beneficial species. As described by Olivier in U.S. Pat. No. 6,780,637 B2, this organism has the ability to rapidly and ravenously consume a wide range of organic materials and convert them into insect matter, leachate and a frass residue that can be used as a fertilizer. The insect matter can be used to feed animals, or separated into its component constituents of protein, oil and chitin.

Growing black soldier flies in captivity is a function of managing the two dynamic stages of its life cycle, the larval stage and the adult stage. The adult stage of the BSF life cycle is very short, and typically lasts for only about 4 days. This stage is concerned only with seeking and securing a mate, mating and laying eggs in a suitable location. Adult flies do not even have complete mouthparts with which to feed. In contrast the larval stage can last from weeks to months, and is spent in the search for and consumption of food. BSF larvae can consume up to twice their own body weight each day. In the process they generate both solid and liquid waste, termed frass and leachate respectively. Thus, the primary tasks surrounding the large-scale culture of BSF larvae consist of devising ways in which to successfully feed the larvae, handle the waste materials that they generate and harvest them at the desired larval or prepupal stage.

Previous methods of growing black soldier fly and other insect larvae have typically involved the use of flat trays or static containers. An example is provided by Newton and Sheppard in US 2013/0319334 A1. Eggs and/or larvae are placed in trays or containers and food is added periodically to the surface of the mass of food and larvae. A hole or screen at the bottom generally serves to drain away the leachate produced. Because BSF larvae must have an adequate supply of air, they tend to stay within about 7 to 10 cm of the surface of the food pile. Excessive amounts of food deprived of exposure to air run the risk of undergoing anaerobic fermentation. Most published reports suggest that BSF larvae tend to avoid areas of anaerobic activity and prefer not feed in them. This is undesirable because it deprives feeding larvae of this material and results in incomplete consumption and utilization of the feedstuff. Anaerobic decomposition can also create foul odors and undesirable byproducts. Conventional flat trays and containers can also experience difficulties with temperature regulation. Both BSF larvae and anaerobic decomposition produce heat. The optimal growth temperature for BSF larvae is 80 degrees to 95 degrees F., (27-35 degrees C.). Larvae will seek to avoid higher temperatures by crawling away if they can, and will begin to die if substantially higher temperatures are maintained for a prolonged period. BSF larvae also tend to be photophobic. Because of this is often desirable to cover open trays to allow larvae to feed on the uppermost levels of the food present.

It would be useful to have a means by which large numbers of insect larvae could be grown at depths exceeding 10 cm by facilitating gas exchange through the pile of larvae and food. This would allow the growth of more larvae within a smaller physical area and minimize the “footprint” required for a given level of production. One possible method for doing this is to force or bubble air through a static larva/food pile; however, this approach may or may not lead to even gas exchange throughout the pile.

A need remains for a better way to propagate insect larvae such as Black Soldier Fly larvae.

### SUMMARY OF THE INVENTION

Here we describe an alternative method that promotes even gas exchange and mixing of the pile through by rotating a larval feeding bin.

A system and method are provided for cultivating and harvesting insect larvae such as that of the Black Soldier Fly. The system comprises a drum or bin that is used as a container for holding and feeding insect larvae. The cross-sectional profile of the drum or bin can have a variety of configurations, ranging from circular (for a cylindrical drum or bin), to having any number of sides (e.g., square, pentagonal, hexagonal, and so on.) The side or back end of the bin is fitted with a segmented hinged or removable cover that can be opened entirely or in part to allow access to the interior of the bin. The front end of the bin is fitted with a “migration path”—a removable, truncated conical or roughly conical end piece with a central hole that allows insect larvae and prepupae to exit from the bin, and provides an opening through which food can be continuously or episodically added to the bin. The migration path can either be attached directly to the bin so that it rotates along with the bin, or mounted on a track and held in place so that it remains in a fixed position relative to the bin as the bin rotates.

The bin can be mounted in a variety of ways that will allow it to rotate freely about its central axis. In one embodiment, the bin can be mounted by means of a central axle that either allows the bin to turn freely or turns the bin as it turns. In a second embodiment, the base or back end of the bin can be bolted to a rotatable plate or mounted within a cradle that turns the bin as it turns. In a third embodiment, the bin can be mounted on rollers with the rollers themselves mounted upon a platform or set of rails. In one embodiment of a configuration utilizing rollers, another roller or set of rollers is mounted on one or more rails in order to prevent the bin from sliding longitudinally along or off of its mount while still allowing it to rotate freely. In different embodiment, one or more sets of rollers placed on the mounting rails and oriented parallel to the bin would rest against a raised rim running around the circumference of the bin, thus keeping the bin from sliding forward or backward. In one embodiment, rotation of the bin itself is accomplished by a motor that directly spins the axle, disk or rollers on which the bin is mounted, either continuously or episodically at a predetermined rate. In a different embodiment an external wheel, gear or belt is used to spin the bin itself. Regardless of the type of mounting or drive used, the bin can be mounted horizontally or at an angle relative to the floor. In another embodiment, one or more bins may be mounted on rails or any other flat surface, and rolled or rotated to and fro by pushing on the sidewall. If multiple bins are mounted adjacent to one another with their sidewalls touching, entire rows of bins may be rolled and rotated by pushing on the outermost bin on one end of the row or the other.

One or more sets of drainage holes can be provided around the circumference of the bin to allow excess liquid (leachate) to egress from the feeder space and be collected by an excess fluid/leachate collection system. These holes can be lined with a screen, perforated plate and/or filter material in order to prevent the escape of small larvae. Optionally, dedicated drainage holes may be omitted entirely and excess leachate can be allowed to accumulate to the level where it is allowed to drain via the migration path(s) present. The interior of the bin may be lined with a series of ridges or blades in order to facilitate mixing of the contents as the bin rotates. If a truncated conical end piece is used, it can be fitted with a set of ridges that can be angled to facilitate the migration of larvae and prepupae from the bin. Larvae and prepupae are allowed to crawl up the inside of the migration path, through the central hole, and thereby fall gravitationally into a larval collection system. Food can be introduced into the open end of the migration path by hand, or via an automated feeder system.

A set of wired or wireless sensors and/or cameras may be mounted on a tube, beam, feeding system or other structure inserted into the bin through the hole in the migration path. These can be used to continuously or intermittently measure and observe parameters including but not limited to temperature, humidity, the extent to which the bin is full and other operating parameters. Likewise, insect eggs may be hung from a structure inserted inside the bin through the hole in the migration path, so that when the eggs hatch new larvae fall directly onto the food and larval contents of the bin. The entire bin and any mounting apparatus may be placed on or hung from a scale in order to measure the weight and any changes in weight.

The system of the present invention is designed to mitigate or eliminate many of the problems encountered by flat tray or container-based systems including systems that are designed to bubble air throughout a food pile. It does this by utilizing the continuous or intermittent rotation of a cylindrical bin to mix and aerate the mass of larvae and food material that it contains. The mixing process distributes oxygen, carbon dioxide, food, water, heat and the larvae themselves more evenly throughout the food/larval mass. It also provides an opportunity for excess heat to dissipate. Many insect larvae are naturally photophobic. Use of a substantially enclosed bin as a growth and feeding chamber automatically provides a dark environment and obviates the need to supply a separate cover.

Embodiments of the current disclosure have the advantage of economies of scale. Since the volume of a cylinder increases according to the square of its radius, cylinders of larger diameters will produce substantially more larvae than cylinders with smaller diameters.

This means that larval production can be increased by using fewer larger bins, thereby reducing the number of feeders, larva collectors, monitors and employees needed to achieve a given level of production.

The foregoing and other objects, features and advantages of the invention will become more readily apparent from the following detailed description of a preferred embodiment of the invention which proceeds with reference to the accompanying drawings.

### BRIEF DESCRIPTION OF THE DRAWINGS

A more complete and thorough understanding of the present embodiments and the advantages thereof may be found by referring to the following descriptions in conjunction with their accompanying drawings. The reference numbers indicate the features indicated in the descriptions.

FIG. 1 illustrates one embodiment of the invention in a frontal oblique view.

FIG. 2 illustrates the same embodiment depicted in FIG. 1, but with a rear oblique view.

FIG. 3A illustrates the same embodiment depicted in FIGS. 1 and 2, but shown from the top view, and FIG. 3B is a cross-sectional view along the axis.

FIG. 4 illustrates an embodiment that is similar to the type of bin depicted in FIGS. 1-3, but with migration paths at both ends.

FIG. 5 illustrates an alternative embodiment of the way in which the rotating larval culture bin is mounted and rotated.

FIG. 6 is a bottom view that illustrates the arrangement of the positioning rollers on the underside in FIG. 4.

FIG. 7 illustrates the flow of material through the bin, starting with food waste, manure or other organic material to be fed to the insect larvae and ending with harvesting the larvae and byproducts.

FIG. 8 illustrates an alternative bin mounting and rotation configuration using a bin that has (as an example) a hexagonal cross section and mounted for rotation by means of an axle positioned along the central axis of the bin.

FIG. 9 illustrates an alternative bin mounting and rotation configuration in which the bin is removably mounted in a rotatable cradle.

### DETAILED DESCRIPTION

The present disclosure relates to systems and methods for producing large numbers of insect larvae—particularly black soldier fly larvae—in a relatively small space and in an automated or semi-automated fashion. Embodiments of the disclosure may include a number of different ways in which to utilize one or more rotating bins or barrels to mix, aerate and separate combinations of food, insect larvae, frass and leachate. They may also include a number of different configurations that allow the entire process to be measured, monitored and controlled in real time.

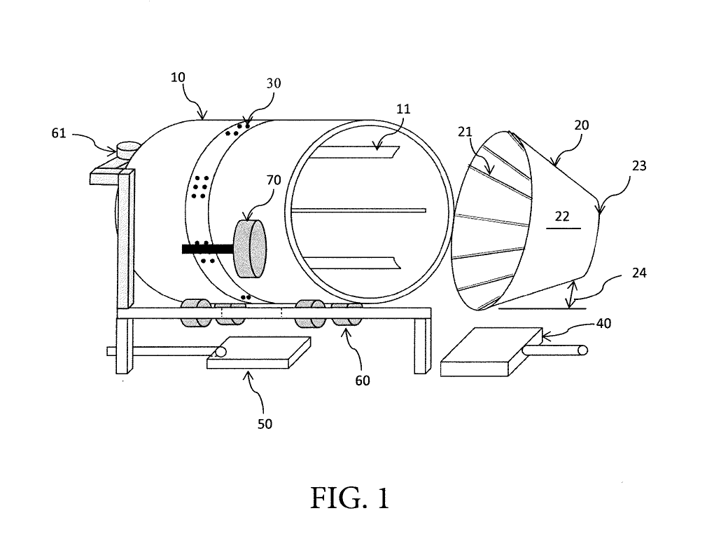

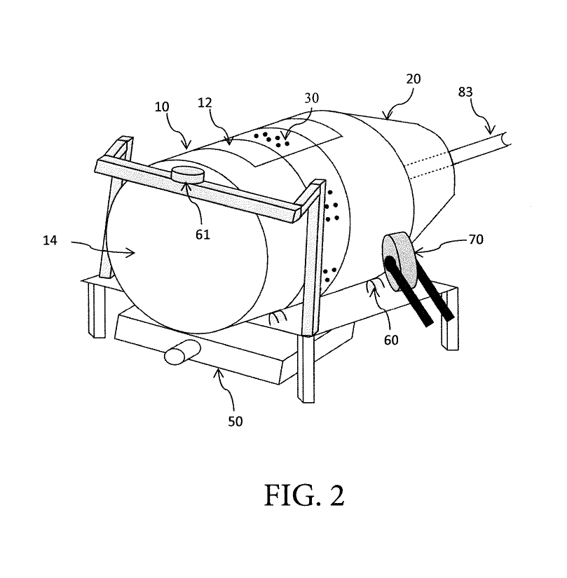

In general, each feeding and cultivation bin will have one or more of the components shown in FIG. 1. These include a rotatable bin, tank or barrel 10 into which food and insect larvae are introduced. The bin sidewall contains a series of holes or slits 30 that are covered with fine cloth, screen, mesh or filter and whose function is to allow excess liquid and leachate to drain from the bin into a leachate collection system. In this embodiment one end 14 (FIG. 2) of the cylinder is closed. Attached to the opposite end is a conical opening structure or “migration path” 20 that with an axial end opening 23 allows for the introduction of food, water and other material into the bin, and egress of mature larvae out of the bin. In this embodiment, the migration path is preferably in the shape of a truncated cone. The incline angle 24 defined by the migration path sidewall 22 is anywhere between 5 degrees and 90 degrees, with the preferred angle of inclination being in the range of 45 degrees to 60 degrees.

The migration path structure itself is shown in a detached configuration as it might be for assembly, cleaning or servicing. A migration path may be mounted in one of three ways. In one embodiment, the migration path is attached to the bin and rotates with it. In a second embodiment, the migration path is mounted on or alongside the bin, but is held in a fixed position and remains motionless as the bin rotates. In a third embodiment, the migration path rotates in the same direction as the bin, but turns at a slower rate. It is preferable although not required that the bin and migration cone be made from food-grade plastic or stainless steel. The inside surface of the bin may be lined with mixing bars, projections or ridges 11 whose function is to mix and aerate the contents of the bin as bin turns.

In this embodiment each bin is mounted upon a set of wheels or rollers 60 that allow the cylinder to spin around its central axis. These wheels or rollers can be powered or unpowered depending upon the particular embodiment of the device. In the particular embodiment depicted in FIG. 1, the rollers themselves are unpowered and an external wheel, belt, or gear 70 is used to turn the bin.

Also present are a leachate collection system 50 that is used to drain and collect excess liquid and leachate produced in the feeding and cultivation process, and a larval collection system 40 that accepts larvae migrating out of the bin and either accumulates them for later collection or actively forwards them to another location for storage or processing.

In addition, a mechanism 61 can be implemented to prevent the bin itself from moving axially forward and/or backward and falling off its mounting. In the embodiment shown, this consists of a set of external braces and rollers that bar the bin from moving forward or backward out of its preferred location and alignment.

In this embodiment, ridges 21 are present on the inside of the migration cone. The function of these ridges is to facilitate the rapid egress of mature larvae out of the bin and into the larva collection system. The feed input system is not shown in this diagram.

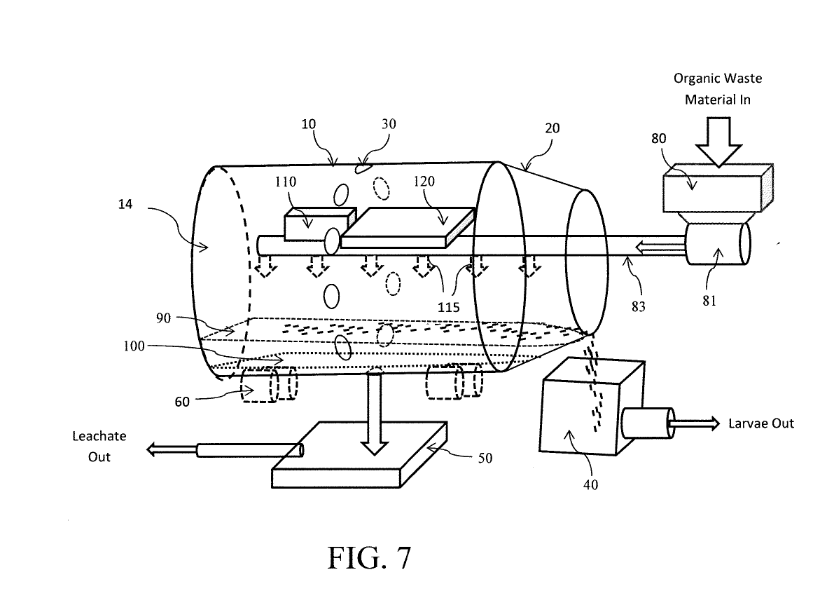

FIG. 2 shows the same embodiment of the invention as in FIG. 1, but from a different view. In this diagram the migration cone is in the attached configuration. The diagram also illustrates additional components of the device. These include a sidewall hatch or opening 12 positioned somewhere on the bin that can be used to load or empty the bin, sample its contents, provide access to heating, cooling and monitoring equipment or clean or inspect the inside. Also shown is a portion of a feed input system 83 consisting of the tube, auger, conveyor or other means that is used to introduce food and serve as a mount for heating, cooling and monitoring equipment located inside the bin. Like the platform or rails on which the bin itself is mounted, the feed input system may be mounted on wheels to facilitate its transportation and positioning. The feed input system is further shown in FIG. 7.

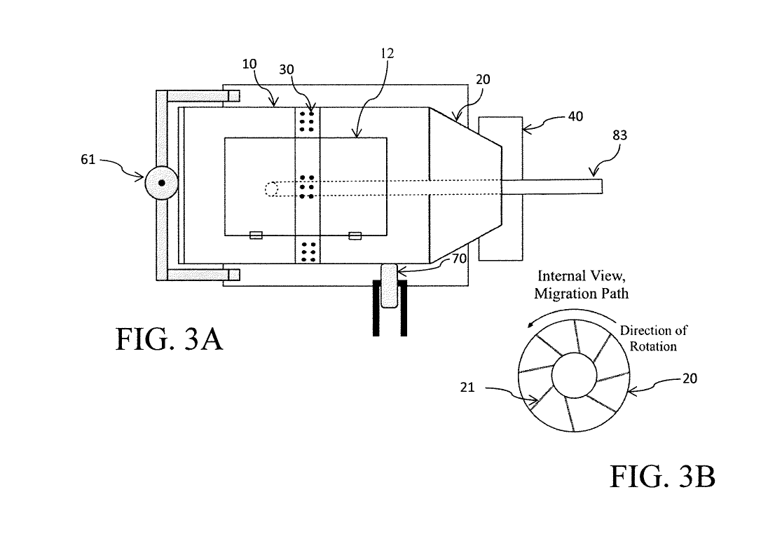

FIG. 3 illustrates the same embodiment depicted in FIGS. 1 and 2, but shown from the top view, and FIG. 3A shows cone 20 in cross-section. The diagram in FIG. 3A illustrates an optional spiral or offset configuration of the internal migration path ridges 21 relative to the direction of rotation of the bin and a migration path 20 that may be rotating along with it. This spiral configuration of ridges is designed to reduce the incline 24 (FIG. 1) that larvae must negotiate as they seek to exit the rotating migration path.

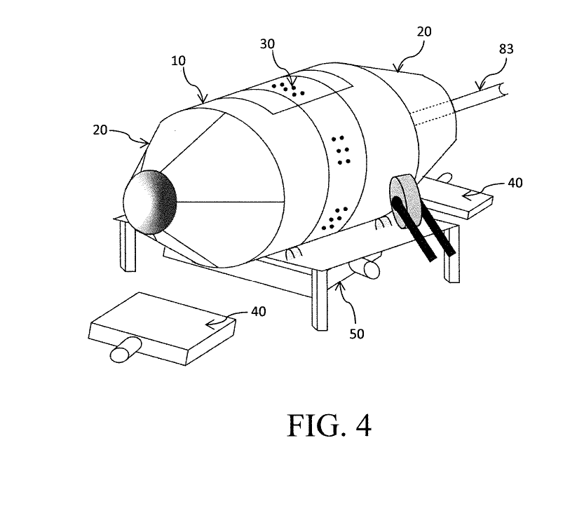

FIG. 4 illustrates a bin configuration that is similar to that shown in FIGS. 1-3, but has a migration path 20 at both ends of the bin. As a result the larvae collection 40 is also present at both ends. A feeding system tube, auger or conveyor 83 may be present at one or both ends as required.

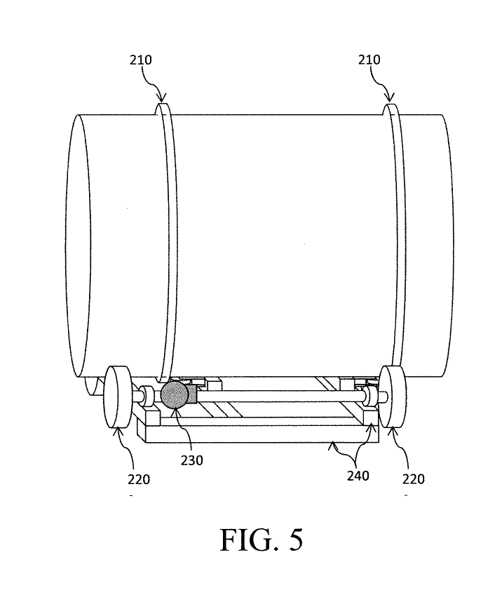

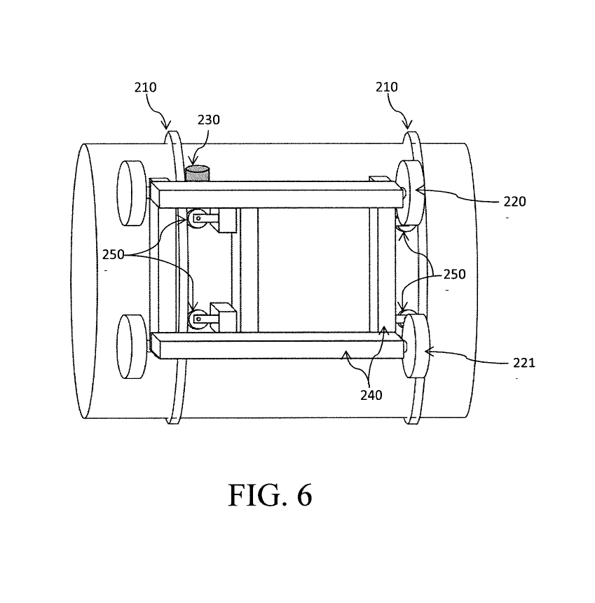

FIG. 5 illustrates an alternative embodiment of the way in which the rotating larval culture bin is mounted and rotated. Rather than using passive rollers and an external wheel, belt or other means of turning the bin, a variable speed motor and gearbox 230 are mounted directly on or adjacent to an axle connecting one or more rollers. This motor is used to turn one or more rollers 220, thereby rotating the bin itself at a desired rate and/or at desired time intervals. Instead of using upright supports to prevent forward and backward migration of the bin, in this figure an embodiment is shown in which positioning rollers 250 (more readily visible in FIG. 6) are attached to the frame of apparatus and mounted either parallel with, or at a slight angle to, the outside lateral surface of the bin. In this position the surface of the rollers come in contact with two raised positioning ridges 210 mounted around the circumference of the bin. Any attempt at forward or backward motion by the bin will be resisted by the pressure of the positioning rollers against the positioning ridges.

FIG. 6 illustrates the arrangement of the positioning rollers 250 on the underside of the embodiment of the invention described in FIG. 5. In this embodiment, rollers on one side of the mount 220 are powered, while rollers on the other side 221 are not. However, any configuration of powered and unpowered rollers may be used.

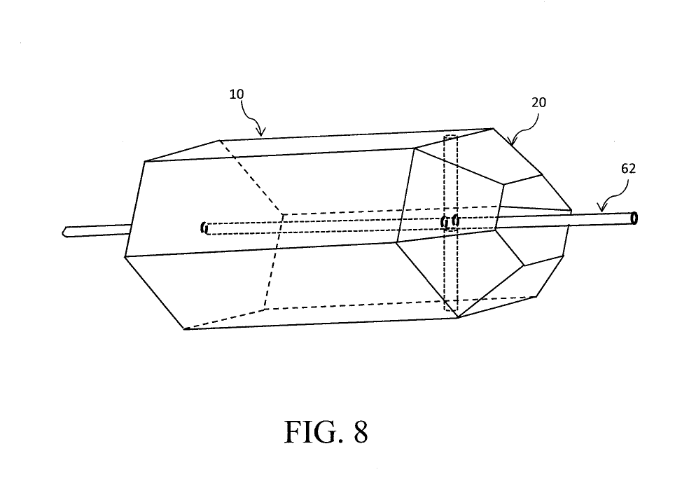

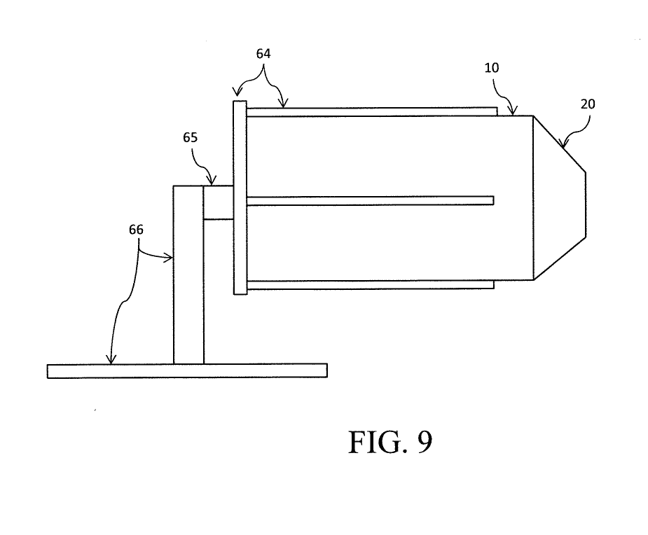

FIG. 7 illustrates the flow of material through the bin, starting with food waste, manure or other organic material to be fed to the insect larvae and ending with harvesting the larvae and byproducts. As shown, the apparatus consists of a bin or barrel 10 on which one or two migration paths 20 are mounted. The bin sidewall is perforated around its circumference with holes, slits or other openings 30 that are screened or filtered to selectively allow excess liquid or leachate within the bin to drain out while simultaneously retaining larvae, food and frass within the bin. The bin may rest upon one or more sets of rollers 60 that allow it to rotate around its central axis in a smooth and controlled fashion as desired by the operator. As described previously, the bin can be turned in one of many different ways, including by use of an external wheel, belt or gear, by powering one or more sets of the rollers upon which the barrel rests, or by using a different mounting configuration as shown in FIG. 8 or FIG. 9.

Food waste, manure or other organic material is screened for undesirable foreign material such as metals and plastics using magnets, sorters or other means and then ground and placed into a silo, hopper or reservoir 80. In another embodiment the feed material can be loaded into the hopper or reservoir in unground form, and is subsequently ground in a combination pump/grinder. The material fed into a pump, auger or pump/grinder 81 is moved through one or more sets of tubes or pipes 83 into the interior of the feeding/culture bin. Once inside the bin, the feed material is extruded or sprayed across the surface of the combined food/larval mass 90. As a result of the action of gravity and the feeding process, excess liquid, leachate and frass will tend to settle and accumulate in a layer toward the bottom 100 of the bin. The action of gravity will cause excess liquid and leachate to filter through the drainage openings 30, and fall into the leachate collection system 50. In one embodiment the leachate collection system simply consists of containers used to collect excess liquid and leachate. In a different embodiment, the leachate collection system would actively or passively pump or drain its contents to a remote location for storage, collection and processing.

Once they are mature, black soldier fly larvae will naturally seek to migrate away from the food pile to a nearby location where they can pupate. Either by instinct, attraction to light or by the process of trial and error, they will eventually crawl up the inside surface of the migration path and subsequently fall into the larva collection system 40. The larva collection system can function in a number of ways. In one embodiment it simply serves as a storage area that is configured in such a way that the larvae accumulate and cannot crawl out. This storage system can subsequently be emptied either manually or automatically into containers for transportation to a different location. In a different embodiment it is part of an active or passive transport system that moves harvested larvae out of the bin area into a separate location for storage, shipment or processing.

Frass that accumulates within the bin must eventually be removed. This can be done in a number of ways. In one embodiment, the accumulated frass can be reached by use of the access hatch shown in FIG. 2 and FIG. 3. In another embodiment, the frass can be accessed by removing the migration cone 20 from the front of the bin. In a third embodiment, a closed end 14 of the bin can be made in such a way that it can be partially or completely removed for cleaning purposes.

Either the feeding system itself or a mounting rail or suspension system that parallels the course of the feed supply system 83 within the bin can be modified for use as a platform that resides within the cavity of the bin. This platform can be used to support a number of useful functions. In one embodiment, this platform is used to support one or more sensors and monitors 110 that are used to sense, record and/or transmit parameters that include but are not limited to temperature, humidity, light level, bin rotation rate, feed monitoring devices and one or more cameras that allow operators to view the interior of the bin in real time. In another embodiment, the platform can be used to support a space 120 used for the placement of insect eggs, larvae, or a group of receptacles designed to serve as an attractive place for adult black soldier flies or other desired insects to lay their eggs. Placing eggs, larvae or egg-laying receptacles in this location is the preferred way of introducing new larvae into the bin, as both adults and larvae will be attracted to the organic feed material 115 as it exits the feeding tubes.

FIG. 8 illustrates that the transverse cross section of the bin 10 need not be cylindrical, but could be of virtually any shape found to minimize cost and maximize some other desired characteristic such as effective volume, mixing, or ease of construction. Likewise, the bin may be mounted parallel to, or at an angle, with respect to the floor. The figure also demonstrates that bin mounting and rotation can be alternatively accomplished by use of a central longitudinal axle 62 that either turns with the bin or allows the bin to turn freely on a set of axle-mounted bearings.

FIG. 9 illustrates yet another method of mounting and rotating the bin that makes use of a mounting plate and/or cradle 64. The bin is mounted to the end plate or inserted into the cradle. The plate or cradle is itself attached to an axle and set of mounting bearings 65 that allow the plate or cradle to be spun by means of gears, belts, wheels or an in-line mounted electric motor. The axle and bearings are themselves attached to a stand or mount 66 that can be floor or wall-mounted.

Having described and illustrated the principles of the invention in various embodiments thereof, it should be apparent that the invention can be modified in arrangement and detail without departing from such principles. I claim all modifications and variations coming within the spirit and scope of the following claims.

The invention claimed is:

1. A system of feeding, culturing and harvesting insects, particularly black soldier flies comprising:
a semi-enclosed barrel including a generally cylindrical sidewall having two ends and a central axis of rotation oriented roughly transversely to the direction of gravity and a migration path attached to one or both ends of the barrel for the exit of mature larvae;
the migration path which has an axial opening at an end of a barrel allowing for the input of feed material and the egress of larvae;
means for supporting the bin transversely to the direction of gravity while allowing it to rotate about its central axis;
means for turning the bin about its central axis;
means for placing feed into the interior of the bin via the migration path;
means for collecting and accumulating larvae that have exited from the bin via the migration path; and
means for collecting and accumulating leachate draining from the bin via the sidewall openings.

2. The system according to claim 1, in which the migration path is configured to create an incline angle with the sides of the barrel ranging from 5 degrees to 90 degrees, and a preferred incline angle of 45 to 60 degrees.

3. The system according to claim 1, with a transverse section of the barrel forming a circle, ellipse, or closed polygon with any number of sides.

4. The system according to claim 1, wherein the barrel is mounted using a mounting axle positioned along the central axis of rotation, a rotatable mounting end plate or cradle, or a system of rollers.

5. The system according to claim 1, including a means for controlling a speed and frequency of rotation of the barrel and wherein the speed and frequency of rotation of the bin are controlled manually or automatically.

6. The system according to claim 1, including a set of ridges on the interior of the migration path arranged in a spiral or offset fashion in order to minimize the amount of climbing larvae must do to exit a rotating migration path.

7. The system according to claim 1, including a set of internal mixing bars or ridges mounted inside the bin to facilitate aeration and mixing of the barrel contents.

8. The system according to claim 1, including a larva collection system positioned under the migration path to capture and accumulate larvae exiting the barrel.

9. The system according to claim 1, including a series of drainage openings distributed around the sidewall to allow for drainage of excess liquid and leachate.

10. The system according to claim 9, wherein screens or filters are affixed to the drainage openings to prevent the egress of material other than excess liquid or leachate.

11. The system according to claim 9, including a leachate collection system positioned under the drainage openings in the barrel to facilitate collection of excess liquid and leachate.

12. The system according to claim 1, including an access hatch to facilitate the loading, unloading, inspection and cleaning of the barrel.

13. The system according to claim 1, in which the means for placing feed is coupled to a feed input system comprising a hopper, silo or other reservoir for containing food waste, manure or other organic matter, a means of removing metal, plastic or other unwanted non-food items, a grinder, or pump capable of moving ground food waste and manure into a series of pipes and tubes or conveyer belts that distribute the feed material into the interior of the barrel.

14. The system according to claim 1, including a means of controlling the rate at which feed is placed into the barrel.

15. The system according to claim 1, including one or more platforms suspended inside the barrel to mount equipment, monitoring devices, controls and sensors as well as eggs, larvae, or receptacles for eggs laid by adult insects.

16. The system according to claim 1, including a system for monitoring contents and environment within the bin, including temperature, humidity, feed flow rate, and viewing the contents.

17. The system according to claim 1, wherein the barrel is rotated by an external wheel, belt or gear, or the barrel is rotated by a motor, belt or gear turning an axle, a device mounted on an axle, a mounting plate or cradle, or one or more rollers.

18. The system according to claim 1, wherein one end of the barrel is closed and the other has an attached migration path in the form of a truncated cone.

19. The system according to claim 1, wherein both ends of the barrel have attached migration paths.

20. The system according to claim 1, in which the migration path or paths are fixed to the barrel and rotate along with it, or do not rotate along with the bin, or rotate at a rate that is slower than the rotation rate of the bin itself.

21. A method of raising insect larvae and byproducts comprising:
inserting larvae or eggs into a bin;
comminuting and inserting food material into the interior of the bin;
aerating the larval/food mass by rotating the bin about a central axis that is positioned roughly transverse to the direction of gravity;
draining leachate from the bin into a collection system;
and providing a migration path for the egress of mature larvae on at least one end of the bin into a larval collection system.
